(19) United States
(12) Patent Application Publication
(10) Pub. No.: US 2025/0205370 A1
(43) Pub. Date: Jun. 26, 2025

(54) SYNTHESIS OF TOLUIDINE BLUE O AND A KIT FOR MUCOSAL APPLICATION

(71) Applicant: Swagatika PANDA, Bhubaneswar (IN)

(72) Inventor: Swagatika PANDA, Bhubaneswar (IN)

(21) Appl. No.: 18/848,369

(22) PCT Filed: Mar. 18, 2023
(86) PCT No.: PCT/IN2023/050263

§ 371 Date: Sep. 18, 2024

(30) Foreign Application Priority Data
Mar. 19, 2022 (IN) 202131053258

(51) Int. Cl. A61K 49/00; A61P 35/00; C07C 381/04; C07D 279/18

(52) U.S. Cl. CPC A61K 49/006; A61P 35/00; C07C 381/04; C07D 279/18

(57) ABSTRACT

The present invention describes synthesis of Toluidine blue O for a formulation to be used for screening and biopsy site identification in oral cancer. The invention discloses two formulations and a method of delivery of the said formulations via a kit. The method described is a reproducible method of synthesis of pharmaceutical grade Toluidine blue O of acceptable purity and good colorimetric value. The present invention also discloses a kit that includes one formulation of a cleaning solution containing an organic acid and another formulation that is a vital stain formulation. The kit is packaged as a dual chamber sprayer having two pump dispensers, that facilitates ready to use, convenient dental chair side delivery, and allows mass screening.

### FIELD OF THE INVENTION

The present invention relates to synthesis of Toluidine blue O, and particularly two formulations and a kit for mucosal application for screening and biopsy site identification in oral cancer.

### BACKGROUND OF THE INVENTION

Oral cancer occurs anywhere in the oral cavity and develops in the tissues and cell linings of the mouth, throat and inside of the cheeks. The cancer may manifest as a sore or ulcer that does not heal, as a lump or as an unusual growth or a patch that stays in the mouth for long. Ignorance on the part of the patient and late diagnosis are the major factors that prevent early detection of the cancer and subsequent poor prognosis. The difficulties involved in the identification of biopsy site in non-healing ulcer complicate the diagnostic process. The International Agency for Research in Cancer and World Health Organization has stressed upon the fact that oral cancer can be managed well by planning effective screening strategies.

Oral cancer can be diagnosed in early stages by implementing successful screening approaches. Many screening tools are available based upon the principle of autofluorescence, chemiluminescence and tissue reflectance of oral mucosa. The huge cost and technical sensitivity involved in these procedures make them practically unsuitable for patients in a developing country like India.

Another technique of screening of oral cancer is intravital staining based on acid-base interaction of basic chemical with nucleic acids of tumour epithelial cells resulting in a colorimetric reaction observable by the naked eyes. Toluidine blue O is one such vital stain used for the detection of cancerous cells in the oral cavity of the subject.

US patent application U.S. Pat. No. 5,372,801A describes a biological stain composition containing Toluidine blue O and pharmaceutically acceptable oxidizing agent. US patent application U.S. Pat. No. 4,321,251A discloses a composition for detecting malignant lesions of oral cavity by a composition of Toluidine blue O in acetic acid solution. The prior art discloses Toluidine blue O stain in combination with oxidizing/buffering agent.

Mashberg et al proposed a protocol for oral cancer screening through vital stains that requires cleaning the surface of the oral lesion by acetic acid followed by topical application of vital stain and removal of the excess stain by acetic acid. The dark blue coloration of the lesion indicates oral cancer. The color value of Toluidine blue O depends upon the purity and nature of isomers. Cumbersome instant preparation of Toluidine blue O solution with the commercially available laboratory grade Toluidine blue powder and its application along with acetic acid in three steps make the screening process inconvenient for the dental surgeon. Toluidine blue O being a dye and owing to the inconvenient method of application, the work area becomes messy during patient handling. Further, Toluidine blue O is not available as a ready to use form besides a dental chair in a palatable and stable form. These problems have not allowed acceptance of Toluidine blue O for oral cancer screening.

Toluidine blue is absorbed into the tumour cell and forms intercalated bond with the nucleus. For the purpose of regulatory approval and acceptable clinical effectiveness, there is a need for a method of synthesis of Toluidine blue O that should be reproducible and the purity of the conformational isomers of Toluidine blue O being at least 90% excluding its derivatives. There is a need of a formulation of this Toluidine blue O for mucosal application in oral cancer screening for the convenience in screening every patient with suspicious lesions and effortless identification of the biopsy site. There is also a need to improve the palatability of the stain formulation, so that the taste of the formulation is acceptable by the patients.

### SUMMARY OF THE INVENTION

The present invention describes a process 100 for the synthesis of Toluidine blue O. The process includes a first stage 105 of synthesizing of 2-amino-5-dimethylaminophenyl thiosulfonic acid, a second stage 110 of synthesizing of indamine thiosulfonic acid, a third stage 115 of synthesizing Toluidine blue O and Toluidine blue O Zinc Chloride Double Salt, and a fourth stage 120 of purifying Toluidine blue O.

The first stage 105 of synthesis of 2-amino-5-dimethylaminophenyl thiosulfonic acid further includes the first step of adding hydrochloric acid to a stirred solution of N, N-dimethyl-1,4-phenylenediamine in water and stirring for 15 mins to obtain a reaction mass of pH 2-3. Next, the second step includes adding a solution of aluminum sulfate hexadecahydrate in water to the above mixture of first step and stirring at a room temperature for 10 mins. The third step includes adding a solution of Zinc chloride in water to the above mixture of second step and stirring at a room temperature for 5 mins, and cooling the reaction mass further to 4° C. The fourth step includes adding a solution of potassium dichromate in water dropwise and stirring for 20 mins, followed by bringing the reaction mass to room temperature. The fifth step is adding a solution of sodium thiosulfate pentahydrate in water at room temperature and stirring for 30 mins. The last step that is the sixth step includes heating the reaction mass to 60° C. and stirring for 5 mins and bringing the reaction again to room temperature.

The second stage 110 of synthesis of indamine thiosulfonic acid includes the first step of adding a solution of o-toluidine in water to the reaction mass obtained in the sixth step of first stage 105 at 10° C. and stirring for 5 mins. The second step includes adding a solution of potassium dichromate in water dropwise, and stirring for 10 mins, followed by stirring the reaction mass at 60° C. for 25 mins.

The third stage 115 of synthesis Toluidine blue O and Toluidine blue O Zinc Chloride Double Salt includes the first step of cooling the reaction mixture, obtained in second step of the second stage 110, to 0° C., followed by adding solution of potassium dichromate in water dropwise to it and stirring for 20 mins. The second step includes adding a solution of potassium dichromate in water dropwise to it and stirring for 20 mins. The third step includes adding a solution of zinc chloride in water to the above mixture obtained in second stage at 10° C. and stirring at room temperature for 20 mins. The fourth step is adding a solution of copper sulfate pentahydrate in water to the reaction mass obtained in above third step and stirring for 15 mins; followed by heating the reaction mass at 60° C.; and at this temperature, adjusting the pH of the reaction mass to 2-3 using sulfuric acid solution. The fifth step includes raising the temperature of the reaction mass to 100° C. and stirring for 35 mins, and after 1 hr, cooling the reaction to 0° C. and keeping for 16 hrs.

The fourth stage 120 of purifying Toluidine blue O includes the first step of filtering the reaction mass obtained in the fifth step of third stage 115, over a Buchner funnel and discarding the filtrate. The second step includes washing the solid cake with aqueous sodium chloride solution and discarding the filtrate. The third step is redissolving the solid residue in 100 ml water and stirring for 4 hrs at room temperature. The fourth step includes heating the reaction at 75° C., and once that temperature is reached, bringing the reaction to 40° C. and stirring for 16 hrs. The fifth step is cooling the reaction, and filtering and discarding the undissolved particles. The sixth step includes adding zinc chloride to the filtrate for precipitation and again cooling to 0° C. The seventh step is redissolving the obtained Toluidine blue O (TBO) in MeOH and precipitating it out with methyl tertbutyl ether as a co-solvent to get TBO. The eighth step includes crystallizing, followed by drying and obtaining a yield of Toluidine blue O as an amorphous powder weighing 0.7 g. The ninth step is characterizing the purity, elution time, area covered and peak intensities of Toluidine blue O and its derivatives by LCMS and NMR.

The present invention further describes a kit for screening and biopsy site identification in oral cancer lesions with convenience that is based on Toluidine blue O synthesized by the process of present invention. The kit includes a first formulation of a food debris cleaning solution containing 1% flavored acetic acid; and a second formulation of Toluidine blue O. The second formulation has Toluidine blue O: 0.5-1 g, Potassium Iodide: 90-100 mg, Ascorbic Acid: 60-65 mg, Acetic Acid: 3-5 g, Ethanol: 7-10 ml, Menthol: 0.2-0.4 g, and Water added to make 100 ml. The formulations are dispensed through a dual chamber sprayer.

The first chamber of the sprayer contains the first formulation and the second chamber contains the second formulation. The first formulation of cleaning solution is sprayed from the first chamber on the surface of the oral lesion for removing any debris. This is followed by spraying the second formulation of vital stain from the second chamber on the surface of the oral lesion. Finally, the first formulation of cleaning solution is sprayed from the first chamber on the surface of the oral lesion for de-staining the normal cells.

The second vital stain formulation of the present invention is stable after three months of storage; and has C15H16N3S([MS]+) of 270.06 tested after three months of storage with the help of ESI Mass analysis.

### BRIEF DESCRIPTION OF DRAWINGS

The objectives and advantages of the present invention will become apparent from the following description read in accordance with the accompanying drawings wherein,

FIG. 1 shows a plurality of steps involved in a process 100 for the synthesis of Toluidine blue O in accordance with the present invention;

FIG. 2a shows characterization of Toluidine blue O by Liquid chromatography Mass spectrometry (LCMS) for demonstration of purity of conformational isomers N-demethylated and N, N-demethylated derivative of Toluidine blue O in accordance with the present invention;

FIG. 2b shows the peak intensities of Toluidine blue O and its derivatives in accordance with the present invention;

FIG. 2c shows NMR spectra of Toluidine blue O in accordance with the present invention;

FIG. 3 shows stability test result via Electrospray ionization Mass spectroscopy (ESI-MS) analysis of the vital stain formulation in accordance with the present invention after 3 months of storage;

FIG. 4 shows the kit having a dual chamber sprayer containing the flavoured 1% acetic acid in one chamber and Toluidine blue O formulation in other chamber in accordance with the present invention;

FIG. 5a shows a complete view of the lid of the dual chamber sprayer in accordance with the present invention;

FIG. 5b shows a frontal view of the lid of the dual chamber sprayer with height of 42 mm in accordance with the present invention;

FIG. 5c shows a longitudinal section view of the lid of the dual chamber sprayer in accordance with the present invention;

FIG. 5d shows a top view of the lid of the dual chamber sprayer in accordance with the present invention;

FIG. 6a shows a complete view of the collar of the dual chamber sprayer in accordance with the present invention;

FIG. 6b shows a top view of the collar of the dual chamber sprayer in accordance with the present invention;

FIG. 6c shows a longitudinal section view of the collar of the dual chamber sprayer in accordance with the present invention;

FIG. 6d shows a frontal view of the collar of the dual chamber sprayer having height 13.9 mm in accordance with the present invention;

FIG. 7a shows a complete view of the separator of the dual chamber sprayer in accordance with the present invention;

FIG. 7b shows a frontal view of the separator of the dual chamber sprayer with detailed dimensions in accordance with the present invention;

FIG. 7c shows a top view of the separator of the dual chamber sprayer in accordance with the present invention;

FIG. 7d shows a longitudinal section view of the separator of the dual chamber sprayer in accordance with the present invention;

FIG. 8a shows a top view of the bottle of the dual chamber sprayer in accordance with the present invention;

FIG. 8b shows a front view of the bottle of the dual chamber sprayer with detailed dimensions in accordance with the present invention;

FIG. 8c shows a complete view of the bottle of the dual chamber sprayer in accordance with the present invention;

FIG. 8d shows a longitudinal section view of the bottle of the dual chamber sprayer in accordance with the present invention;

FIG. 9a shows a top view of the gasket of the dual chamber sprayer in accordance with the present invention;

FIG. 9b shows a complete view of the gasket of the dual chamber sprayer in accordance with the present invention;

FIG. 9c shows a longitudinal section view of the gasket of the dual chamber sprayer in accordance with the present invention;

FIG. 10 demonstrates positive result after screening, appreciated as dark blue color stained tissue after the application of the two formulations from the kit in accordance with the present invention; and

FIG. 11 demonstrates negative result after screening, appreciated as absence of dark blue color stained tissue after the application of the two formulations from the kit in accordance with the present invention.

### DESCRIPTION OF THE INVENTION

References in the specification to “one embodiment” or “an embodiment” means that a particular feature, structure, characteristic, or function described in connection with the embodiment is included in at least one embodiment of the invention. The appearances of the phrase “in one embodiment” in various places in the specification are not necessarily all referring to the same embodiment.

References in the specification to “preferred embodiment” means that a particular feature, structure, characteristic, or function described in detail thereby omitting known constructions and functions for clear description of the present invention.

The foregoing description of specific embodiments of the present invention has been presented for purposes of illustration and description. They are not intended to be exhaustive or to limit the present invention to the precise forms disclosed and obviously many modifications and variations are possible in light of the above teaching.

The present invention relates to a method of synthesizing Toluidine blue O to achieve an acceptance degree of purity to obtain intense colorimetric reaction when applied on oral cancerous lesions. The method described in this invention is easy to reproduce in manufacturing pharmaceutical grade Toluidine blue O and the achievable purity shall be well accepted by regulatory bodies. The present invention also provides a method of characterization of the obtained Toluidine blue O compound.

The present invention also discloses two formulations including a formulation of Toluidine blue O synthesized by the above said method and a formulation of acetic acid. Both of these formulations are used for screening and biopsy site identification in oral cancer. The present invention also relates to a method of mucosal application of the said formulations.

The foregoing objects of the present invention are accomplished and the problems and shortcomings associated with the prior art, techniques and approaches are overcome by the present invention as described below in the preferred embodiments.

All materials used herein were commercially purchased as described herein or prepared from commercially purchased materials as described herein.

In one aspect, the present invention provides a method of synthesis of pharmaceutical grade Toluidine blue O of acceptable purity and good colour value for a vital stain formulation.

In another aspect, the present invention provides characterization of the Toluidine blue O and its derivatives by Nuclear Magnetic Resonance (NMR) and ESI-MS.

In another aspect, this invention also describes a vital stain formulation, derived from Toluidine blue O as synthesized in the above mentioned procedure. The stability studies for the same are indicated in Example 8.

In an aspect, the invention provides a kit having a vital stain formulation containing Toluidine blue O, and a food debris cleaning formulation, for screening and biopsy site identification in oral cancer lesions. In yet another aspect, the present invention also provides a kit for mucosal application of these formulations by a dual chamber sprayer.

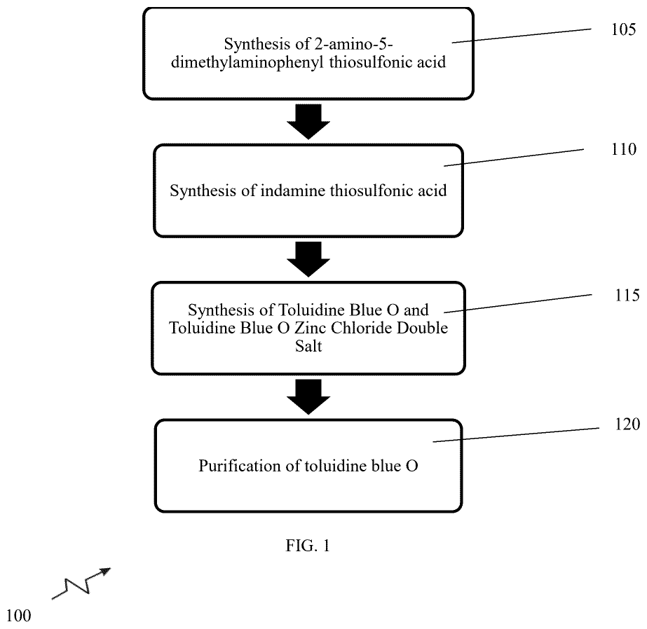

Now referring to FIG. 1, a process 100 for the synthesis of Toluidine blue O is described. The process 100 includes a first stage 105 of synthesis of 2-amino-5-dimethylaminophenyl thiosulfonic acid. This is followed by a second stage 110 of synthesis of indamine thiosulfonic acid. The third stage 115 includes synthesis of Toluidine blue O and Toluidine blue O Zinc Chloride Double Salt. The last stage 120 that is the fourth stage includes purification of Toluidine blue O.

The first stage 105 of synthesis of 2-amino-5-dimethylaminophenyl thiosulfonic acid includes a first step of adding strong acid to a stirred solution of N, N-dimethyl-1,4-phenylenediamine in water and stirring for a first predefined time. The pH of the reaction mass obtained is acidic. In the second step, a solution of aluminum sulfate hexadecahydrate in water is added to the above mixture of first step and stirred at a first predefined temperature for a second predefined time. In the third step, a solution of Zinc chloride in water is added to the above mixture of second step and stirred at a second predefined temperature for a third predefined time. The reaction mass is further cooled to a third predefined temperature.

In the next step that is the fourth step, a solution of potassium dichromate in water was added dropwise and stirred for a fourth predefined time. The reaction mass is then brought to a fourth predefined temperature. In the fifth step, a solution of sodium thiosulfate pentahydrate in water is added at a fifth predefined temperature and stirred for a fifth predefined time. After that, in the sixth step, the reaction mass is heated to a sixth predefined temperature and stirred for sixth predefined time and reaction is brought again at a seventh predefined temperature.

In the first step of first stage, the strong acid is hydrochloric acid and the first predefined time is 15 mins. The pH of the reaction mass is 2 to 3. In the second step, the first predefined temperature is room temperature and the second predefined time is 10 mins. In the third step, the second predefined temperature is room temperature and the third predefined time is 5 mins. The third predefined temperature is 4° C. In the fourth step, the fourth predefined time is 20 mins and the fourth predefined temperature is room temperature. In the fifth step, the fifth predefined temperature is room temperature and the fifth predefined time is 30 mins. In the sixth step, the sixth predefined temperature is 60° C., the sixth predefined time is 5 mins and the seventh predefined temperature is room temperature.

The second stage 110 of synthesis of indamine thiosulfonic acid includes a first step of adding a solution of o-toluidine in water to the reaction mass obtained in the sixth step of first stage at a first predefined temperature and stirred for a first predefined time. The second step includes adding a solution of potassium dichromate in water dropwise and stirring for a second predefined time. After addition, the reaction mass is stirred at a second predefined temperature for a third predefined time.

In the first step of the second stage, the first predefined temperature is 10° C. and the first predefined time is 5 mins. In the second step, the second predefined time is 10 mins. The second predefined temperature is 60° C. and the third predefined time is 25 mins.

The third stage 115 of synthesis of Toluidine blue O and Toluidine blue O Zinc Chloride Double Salt includes a first step of cooling the reaction mixture obtained in second step of the second stage to a first predefined temperature. This is followed by adding solution of potassium dichromate in water dropwise to it and stirring for a first predefined time. In the second step, again a solution of potassium dichromate in water is added dropwise to it and stirred for a second predefined time. In the third step, a solution of zinc chloride in water is added to the above mixture obtained in second step at a second predefined temperature and stirred at a third predefined temperature for a third predefined time.

In the next step that is the fourth step of third stage, a solution of copper sulfate pentahydrate in water is added to the rection mass obtained in above third step and stirred for a fourth predefined time. The reaction mass is heated at a fourth predefined temperature. At the fourth predefined temperature, the pH of the reaction mass is adjusted to acidic using acid solution. In the last step that is the fifth step, the temperature of the reaction mass is raised to a fifth predefined temperature and stirred for a fifth predefined time. Then after a sixth predefined time, the reaction is cooled to a sixth predefined temperature and kept for a seventh predefined time.

In the first step of the third stage, the first predefined temperature is 0° C. and the first predefined time is 20 mins. In the second step, the second predefined time is 20 mins. In the third step, the second predefined temperature is 10° C. and the third predefined temperature is room temperature and the third predefined time is 20 mins. In the fourth step, the fourth predefined time is 15 mins and the fourth predefined temperature is 60° C. The pH of the reaction mass is 2-3 and the acid solution is sulphuric acid solution. In the fifth step, the fifth predefined temperature is 100° C. and the fifth predefined time is 35 mins. In the sixth step, the sixth predefined time is Ihr, the sixth predefined temperature is 0° C. and the seventh predefined time is 16 hrs.

The fourth stage 120 of purification of Toluidine blue O includes a first step of filtering the reaction mass obtained in the fifth step of third stage, over a Buchner funnel and discarding the filtrate. The second step includes washing the solid cake with aqueous salt solution and discarding the filtrate. The third step includes redissolving the solid residue in water and stirring for a first predefined time for first predefined temperature. The next step that is the fourth step includes heating the reaction at a second predefined temperature and once that temperature is reached, the reaction is brought to a third predefined temperature and stirred for a second predefined time.

The fourth stage further includes a fifth step of cooling the reaction and filtering and discarding the undissolved particles. The sixth step includes adding zinc chloride to the filtrate for precipitation and again cooling to a fourth predefined temperature. The seventh step includes re-dissolving the obtained Toluidine blue O (TBO) in alcohol and precipitating it out with a co-solvent to get TBO. The eighth step includes crystallization followed by drying and obtaining the yield. The ninth step includes characterizing the purity, elution time, area covered and peak intensities of Toluidine blue O and its derivatives by LCMS and NMR.

In the second step of the fourth stage, the aqueous salt solution is aqueous sodium chloride solution. In the third step, the first predefined time is 4 hrs and the first predefined temperature is room temperature. In the fourth step, the second predefined temperature is 75° C., the third predefined temperature is 40° C. and the second predefined time is 16 hrs. In the sixth step, the fourth predefined temperature is 0° C. In the seventh step, the alcohol is MeOH and the cosolvent is Methyl tertbutyl ether. In the eighth step, the yield of Toluidine blue O as an amorphous powder weighing 0.7 g is obtained.

For regulatory approval of Toluidine blue O as a drug, ratio of the conformational isomers to that of the demethylation derivatives should be at least 6:1. The Toluidine blue O synthesized by the method of the present invention has a ratio of 14:1.

In accordance with a preferred embodiment, the formulations for screening and biopsy site identification in oral cancer lesions include:
1. A first formulation including a food debris cleaning solution containing an organic acid; and
2. A second formulation including a vital stain formulation.

In this embodiment, the first formulation that is the food debris cleaning solution is 1% flavored acetic acid.

In this embodiment, the second vital stain formulation having Toluidine blue O synthesized by the preferred process of the present invention includes:
1) Toluidine blue O: 0.5-1 g,
2) Potassium Iodide: 90-100 mg,
3) Ascorbic Acid: 60-65 mg,
4) Acetic Acid: 3-5 g,
5) Ethanol: 7-10 ml,
6) Menthol: 0.2-0.4 g, and
7) Water added to make 100 ml.

In a preferred embodiment, the second vital stain formulation includes:
1) Toluidine blue O-1.0 g,
2) Potassium Iodide-100 mg,
3) Ascorbic Acid-63 mg,
4) Acetic Acid-4.610 g,
5) Ethanol-7.48 ml,
6) Menthol-0.3 g, and
7) Water added to make 100 ml.

In accordance with this embodiment, the method of preparation of the second formulation that is the vital stain formulation includes adding Toluidine blue O (1.0 g), potassium iodide (100 mg), menthol (0.3 g), and ascorbic acid (63 mg) in a 200 ml round bottom flask containing 50 ml of deionized water and stirring for 30 mins at room temperature. In the next step, acetic acid (4.4 ml) and ethanol (7.48 ml) are gradually added to the mixed solution and stirred for 15 mins. Next, deionized water is added to achieve a final volume solution of 100 ml. The developed blue-colored slurry is stored in a cool and dry place for future use.

As leuco form of Toluidine blue O stains the tissue better than chromo form, Potassium iodide is added to maintain the leuco form. In addition, potassium iodide kills bacteria that can get into the vital stain formulations. Potassium Iodide also acts as a stain enhancer. The acetic acid is the buffering agent. Ascorbic acid is an anti-oxidant that is used to prevent the dimerization and maintain the color intensity of Toluidine blue O. Ethanol is added as a solvent to improve penetration of the dye into the epithelial tissue. It does not appreciably interfere with the tissue staining mechanism.

The vital stain formulation including Toluidine blue O is formulated with additives that lead to increased shelf life and improvement in other properties. The stain enhancer Potassium Iodide enhances the deep blue colour and increases the cell membrane permeability. Ascorbic acid prevents possible reduction of Toluidine blue O as well as maintains the pH so that the Toluidine blue O remains in the oxidized state. Further, ascorbic acid protects Toluidine blue O from reactive oxygen species (ROS) and irreversible dimerization. Acetic acid acts as a buffering agent to maintain the pH between 3.5-5. Ethanol improves penetration of the dye into the epithelial cell membrane.

Before and after the topical application of the second formulation that is the vital stain formulation, acetic acid that is the food debris cleaning solution has to be topically sprayed. Flavoring of acetic acid is carried out for enhancing the palatability of the cleaning solution to prevent gagging in patients. Menthol flavour is added to 1% acetic acid to enhance the palatability of the cleaning solution.

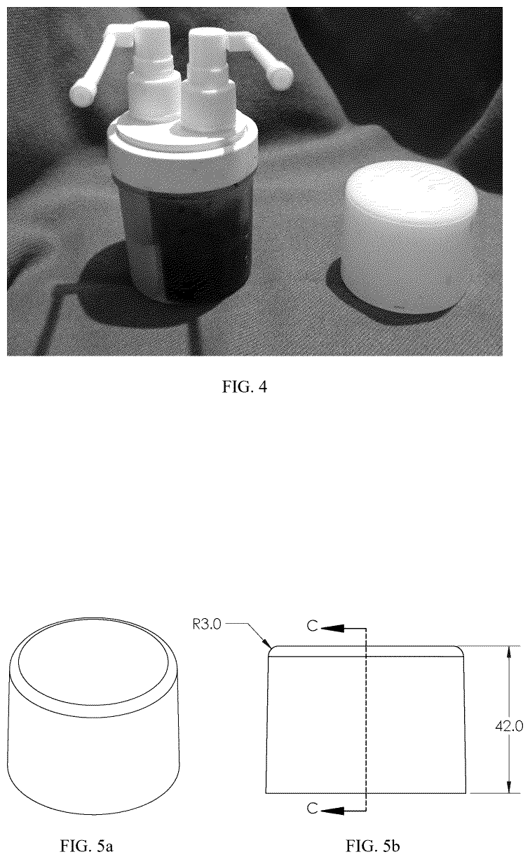

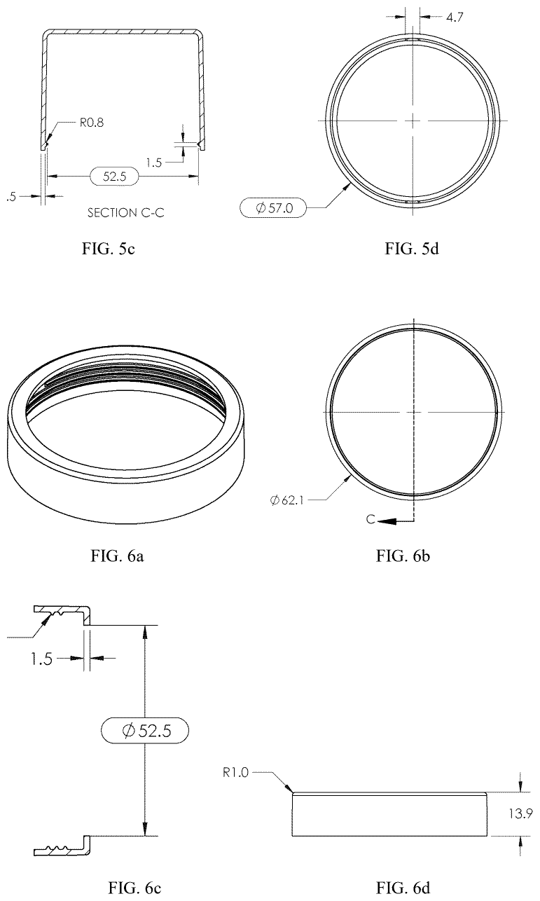

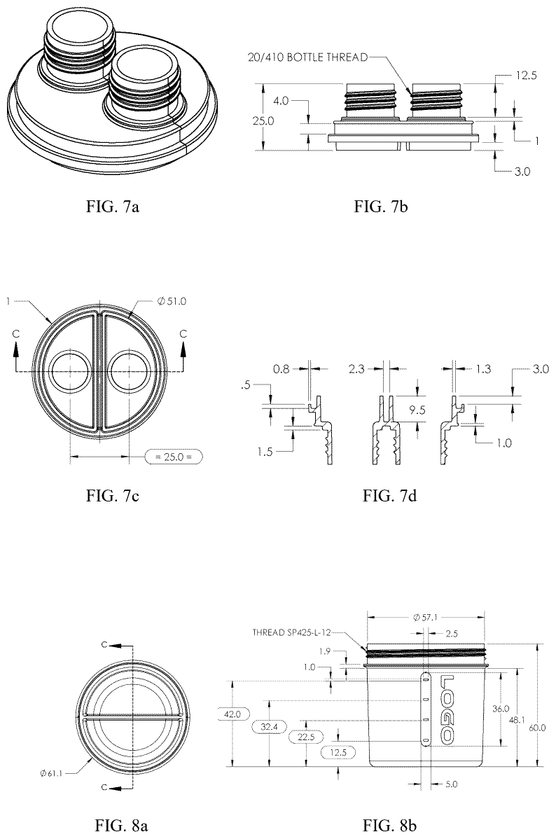

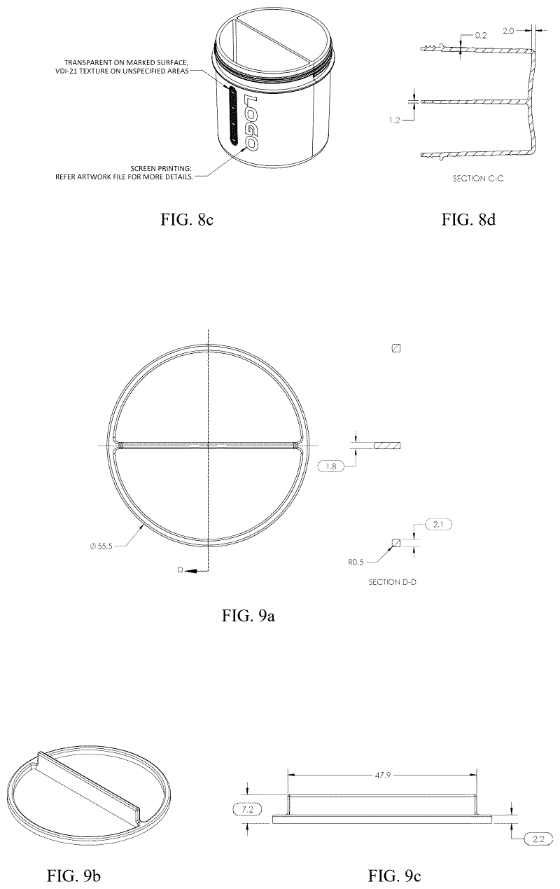

Now referring to FIG. 4, a kit for mucosal application in accordance with the present invention includes a dual chamber sprayer containing the formulations. The two formulations of the present invention are in liquid form and are packaged in the dual chamber sprayer having two pump dispensers, wherein the first chamber includes first formulation of cleaning solution and the second chamber includes the second formulation that is the vital stain formulation. The kit is particularly designed for ready to use, convenient dental chair side setting, and allows mass screening.

Now a preferred method of delivery for the formulations via the kit in accordance with the present invention is disclosed. The method of delivery includes the steps of:
(a) spraying the first formulation of cleaning solution from the first chamber on the surface of the oral lesion for removing any debris;
(b) spraying the second formulation of vital stain from the second chamber on the surface of the oral lesion;
(c) spraying the first formulation of cleaning solution from the first chamber on the surface of the oral lesion for de-staining the normal cells.

In this embodiment, the first step includes spraying 1% acetic acid for cleaning the surface of ulcer. The second step includes spraying the vital stain formulation. The formulation stains the ulcer in blue color. In the third step, 1% acetic acid is sprayed again on the ulcer to remove or destain the blue color from the non-malignant cells so that only the malignant cells retain the stain.

In accordance with a preferred embodiment, the dual chamber sprayer for dispensing the formulations of the present invention further consists of:
a) a lid,
b) a collar,
c) a gasket,
d) a separator,
e) a bottle, and
f) a sprayer head.

Table 1 indicates the specifications for each part of the dual chamber sprayer in the kit of the present invention.

TABLE 1
Specifications for each part of the dual chamber sprayer (all measurements are in millimeters).

| PART | | | | | | WEIGHT | MFG | SECONDARY |
|---|---|---|---|---|---|---|---|---|
| NAME | QTY | TYPE | MATERIAL | COLOUR | FINISH | (grams) | METHOD | OPERATIONS |
| Lid (A) | 1 | Custom | PP | White | SPI-A2 | 16.5 | Injection | |
| | | | | | (Glossy) | | Molding | |
| Collar (B) | 1 | Custom | PP | White | SPI-A2 | 5.84 | Injection | |
| | | | | | (Glossy) | | Molding | |
| Separator (C) | 1 | Custom | PET | White | SPI-A2 | 14.05 | Injection | |
| | | | | | (Glossy) | | Molding | |
| Bottle (D) | 1 | Custom | PET | Transparent | Transparent on | 44.57 | Injection | Screen Printing |
| | | | | | Marked Surface, | | Molding | Colour: Jet |
| | | | | | VDI-21 Texture On | | | Black 19-0303TPX |
| | | | | | Unspecified areas | | | |
| Gasket (E) | 1 | Custom | EPDM Rubber, | White | Natural | 1.64 | Injection | |
| | | | Shore 60A | | | | Molding | |
| Sprayer (F) | 2 | Standard | PP | White | SPI-A2 | | NA | |
| | | | | | (Glossy) | | | |

The sprayer head with nozzle extension, used in this sprayer is a readily available one having following specifications: Diameter-2.5 cms, Height-11 cms, Diameter of nozzle-6 mm, and Height of nozzle extension-6.7 cms.

These and other embodiments will be apparent to those of skill in the art and others in view of the following detailed description of some embodiments. It should be understood, however, that this summary, and the detailed description illustrate only some examples of various embodiments, and are not intended to be limiting to the invention as claimed.

### Examples

Only a few examples and implementations are disclosed. Variations, modifications, and enhancements to the described examples and implementations and other implementations can be made based on what is disclosed.

Examples are set forth herein below and are illustrative of different amounts and types of reactants and reaction conditions that can be utilized in practicing the disclosure. It will be apparent, however, that the disclosure can be practiced with other amounts and types of reactants and reaction conditions than those used in the examples, and the resulting devices various different properties and uses in accordance with the disclosure above and as pointed out hereinafter.

### Example 1: Synthesis of 2-amino-5-dimethylaminophenyl thiosulfonic acid

To a stirred solution of N, N-dimethyl-1,4-phenylenediamine (1.0 g, 7.3 mol, 1.0 eq) in 40 ml water, hydrochloric acid (6 N, 1.57 ml) was added and stirred for 15 min. The pH of the reaction mass was 2-3. The solution of Aluminum sulfate hexadecahydrate (2.5 g, 3.96 mol, 0.6 eq) in 3 ml of water was added to the above mixture and stirred at room temperature for 10 minutes. The solution of Zinc chloride (5.1 g, 36.5 mol, 5 eq) in 3 ml of water was added to the above mixture and stirred at room temperature for 5 min. The reaction mass was cooled to 4° C.

A solution of Potassium dichromate (0.77 g, 2.59 mol, 0.35 eq) in 9 ml of water was added dropwise to the reaction mass and stirred for 20 min. The reaction mass was then brought to room temperature.

To the reaction mass, a solution of Sodium thiosulfate pentahydrate (2.1 g, 13.2 mol, 1.9 eq) in 3 ml of water was added at room temperature and stirred for 30 minutes. After that, the reaction mass was heated to 60° C. and stirred for 5 min and the reaction was brought again to room temperature to obtain 2-amino-5-dimethylaminophenyl thiosulfonic acid.

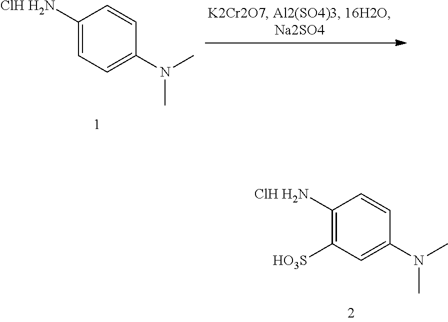

### Example 2: Synthesis of Indamine Thiosulfonic Acid

To the 2-amino-5-dimethylaminophenyl thiosulfonic acid obtained, a solution of o-toluidine (0.8 g, 5.7 mol, 0.8 eq) in 3 ml of water was added at 10° C. and stirred for 5 min.

A solution of Potassium dichromate (0.77 g, 2.59 mol, 0.35 eq) in 9 ml of water was added dropwise to the reaction mass and stirred for 10 min. After addition, the reaction mass was stirred at 60° C. for 25 min to obtain Indamine Thiosulfonic Acid.

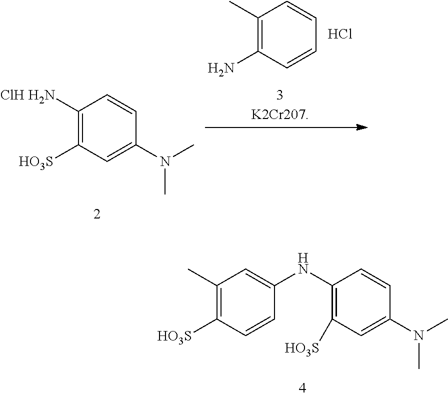

### Example 3: Synthesis of Toluidine blue O and Toluidine blue O Zinc Chloride

### Double Salt

The reaction mixture of Indamine Thiosulfonic Acid was cooled to 0° C. and a solution of potassium dichromate (0.8 g, 5.7 mol, 0.8 eq) in 9 ml of water was added dropwise and stirred for 20 min. Again, solution of potassium dichromate (0.8 g, 5.7 mol, 0.8 eq) in 9 ml of water was added dropwise to the reaction mass and stirred for 20 min.

A solution of Zinc chloride (5.1 g, 36.5 mol, 5 eq) in 3 ml of water was added to the above mixture at 10° C. and stirred at room temperature for 20 min. A solution of copper sulfate pentahydrate (0.7 g, 4.5 mol, 0.6 eq) in 3 ml of water was added to the reaction mixture and stirred for 15 minutes. The reaction mass was heated at 60° C. At this temperature, the pH of the reaction mass was adjusted to 2-3 using sulfuric acid solution. After that, the temperature of the reaction mass was raised to 100° C. and stirred for 35 min. After 1 h, the reaction was cooled to 0° C. and kept for 16 hrs to obtain Toluidine blue O.

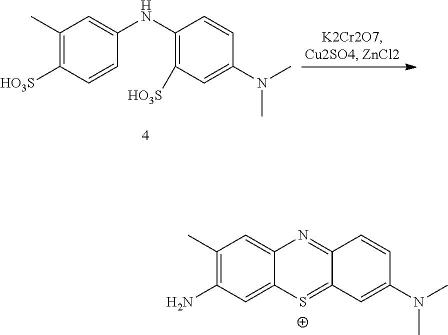

### Example 4: Purification of Toluidine blue O

The reaction mass of Toluidine blue O was filtered over Buchner funnel and the filtrate was discarded. The solid cake was washed with 15% aqueous solution of sodium chloride (100 ml) and the filtrate was discarded. The solid residue was re-dissolved in 100 ml of water and stirred for 4 hrs at room temperature. After that, the reaction mass was heated at 75° C. and once the temperature was reached, the reaction mass was brought to 40° C. and stirred for 16 hr. The reaction mass was cooled and filtered, and the undissolved particles were discarded. To the filtrate, zinc chloride was added for precipitation and again cooled to 0° C.

Finally, the TBO was re-dissolved in MeOH and precipitated out with Methyl tertbutyl ether as co-solvent to get TBO. The precipitate was filtered, dried and ground to obtain 50% yield.

### Example 5: Characterization of Toluidine blue O

The characterization of Toluidine blue O was performed in LCMS, ESI-MS and NMR. The LCMS was undertaken in Quattro Premier XE Micromass MS Technologies. The NMR spectra was recorded using Avance NeoNanobay 400 MHz.

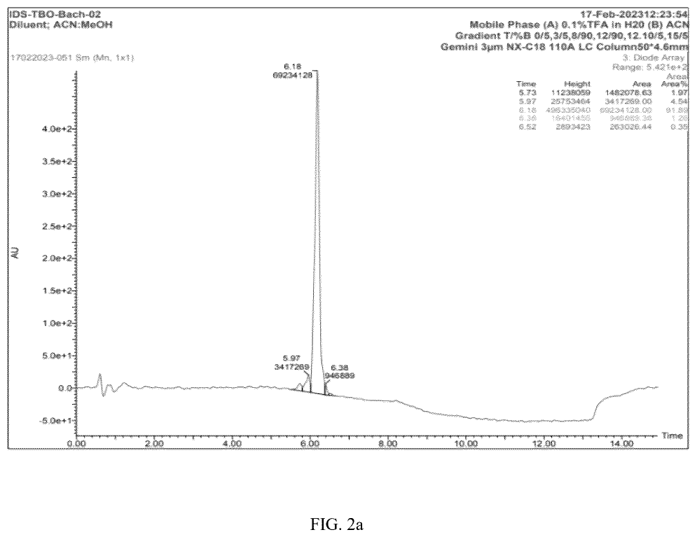

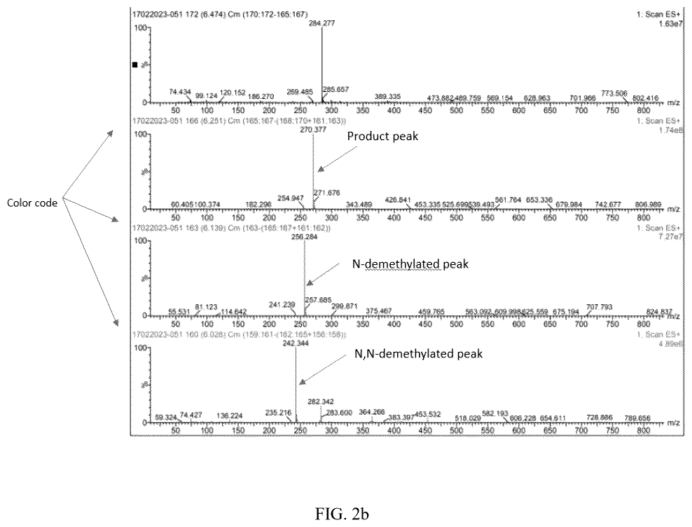

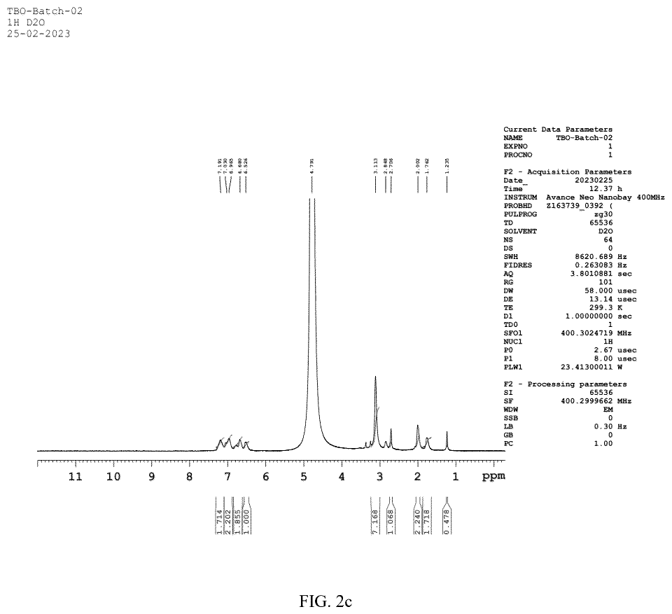

FIG. 2a shows characterization of Toluidine blue O by Liquid chromatography Mass spectrometry (LCMS). The LCMS demonstrated purity of conformational isomers of Toluidine blue O that are N-demethylated and N, N-demethylated derivative as 92%, 4.54% and 1.97% respectively. The remaining 1.60% are unidentified impurities. The elution time, height and area of the peaks of each component are provided in FIG. 2a. The X-axis represents the intensity of the peak while Y-axis represents the retention time of the peak.

FIG. 2b shows the peak intensities of Toluidine blue O and its derivatives. The LCMS demonstrated peak intensities of conformational isomer of TBO (270.38) and its derivatives such as N-demethylated (256.29) and N, N-demethylated (242.34) respectively. The color codes in LCMS correlate with the time compound eluted from the column.

Referring to FIGS. 2a and 2b, the present invention demonstrated a single tall peak that signifies presence of pure substance. The peak corresponds to the mass to charge ratio of the ionized molecule. Height of the peak corresponds to the abundance of ions with that m/z value. There are two small peaks signifying N and N, N demethylated derivatives. Referring to FIG. 2b, the peak intensities of conformational isomer of TBO (270.38) and its derivatives such as N-demthylated (256.29) and N, N-demethylated (242.34) refer to mass of the three molecules.

FIG. 2c shows NMR spectra of Toluidine blue O indicating that there are 6 signals with chemical shifts as δ 7.25 (m, 2H), 7.08 (m, 2H), 6.82 (m, 2H), 6.57 (m, 1H), 3.14 (s, 6H), 2.04 (s, 3H). Referring to FIG. 2c, the HNMR 400 MHz D20 spectra showed following observations.

Number of signals: 6 signals

Chemical shift: δ 7.25 (m, 2H), 7.08 (m, 2H), 6.82 (m, 2H), 6.57 (m, 1H), 3.14 (s, 6H), 2.04 (s, 3H).

### Example 6: Food debris cleaning solution and Vital Stain Formulation

### First Formulation: Food Debris Cleaning Solution.

1 gm of glacial acetic acid was weighed using a balance. Water was added to make the volume 100 ml. The solution was stirred until acetic acid is completely dissolved to prepare 1% acetic acid solution. 0.3 gm Menthol flavor was added in 100 ml of 1% acetic acid solution.

It is applied twice during the test. First, it is applied to clean the food debris from surface of the oral lesion before applying the second formulation. Second, it is applied to remove excess of second formulation from the lesion.

### Second Formulation: Vital Stain Formulation

1) Toluidine blue O-1.0 g,
2) Potassium Iodide-100 mg,
3) Ascorbic Acid-63 mg,
4) Acetic Acid-4.610 g,
5) Ethanol-7.48 ml,
6) Menthol-0.3 g, and
7) Water added to make 100 ml.

Toluidine blue O (1.0 g), potassium iodide (100 mg), menthol (0.3 g), and ascorbic acid (63 mg) was added in a 200 ml round bottom flask containing 50 ml of deionized water and stirred for 30 min at room temperature. In the next step, acetic acid (4.4 ml) and ethanol (7.48 ml) was gradually added to the mixed solution and stirred for 15 min. Next, deionized water was added to achieve a final volume solution of 100 ml. The developed blue-colored slurry was stored in a cool and dry place for future use.

### Example 7: Protocol of spraying of the two formulations via Dual Chamber sprayer

The kit contains a dual chamber sprayer having two formulations, the first formulation that is the food debris cleaning solution in first chamber, and the second formulation that is the vital stain formulation in second chamber.

The protocol of spraying the two formulations via the kit included:
a) spraying the first formulation of cleaning solution from the first chamber on the surface of the oral lesion for removing the debris;
b) spraying the second formulation of vital stain from the second chamber on the surface of the oral lesion; and
c) spraying the first formulation of cleaning solution from the first chamber on the surface of the oral lesion for de-staining the normal cells.

### Example 8: Study of Stability of Vital Stain Formulation

The stability test measurement of the vital stain formulation was done with the help of ESI Mass spectroscopy analysis. On the first day, 1 ml of the blue-colored slurry that is the vital stain formulation having Toluidine blue O was taken for analysis. The slurry was tested using a standard filtration process (used by the mass operator), and the data was collected with the respective instruments. C15H16N3S([MS]+) was calculated as 270.059.

After three months, the same blue-colored slurry that is the vital stain formulation was taken for analysis. The stability of the formulation was tested with the help of ESI Mass analysis. C15H16N3S([MS]+) was found to be 270.06.

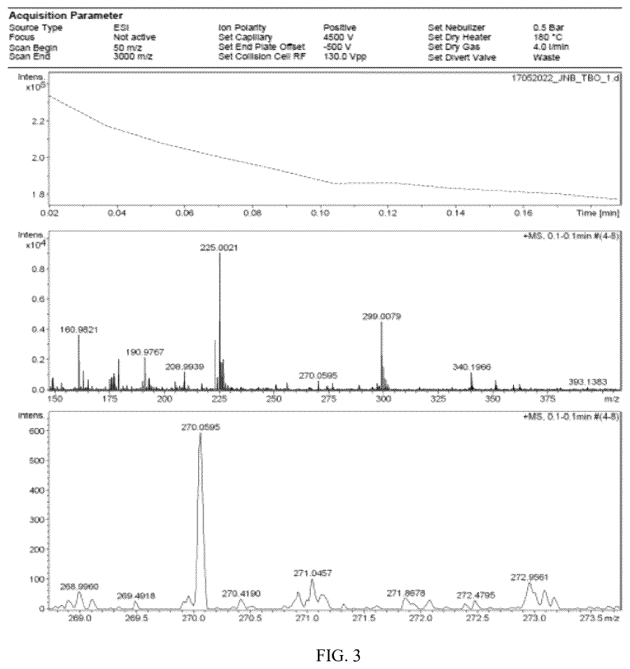

FIG. 3 shows stability test result of the formulation. FIG. 3 demonstrates the Electrospray ionization Mass spectroscopy (ESI-MS) analysis of the vital stain formulation in accordance with the present invention after 3 months of storage. This result signifies that this formulation is unaffected by environmental changes and it can be used as a ready to use oral cancer screening drug.

### Example 9: Study for Assessing Clinical Effectiveness of Sequential Application of Two Formulations Via the Kit in Identifying Biopsy Site and Screening Oral Cancer

1) Patients presenting with non-healing ulcer were seated on the dental chair and draped with aprons to protect their clothes from staining.
2) A thorough clinical examination of the ulcer was performed by inspection and palpation. Clinical signs of the ulcer were recorded.
3) A generous amount of acetic acid was sprayed from the chamber one containing it to clean the surface debris off the ulcer.
4) The vital stain formulation is sprayed from the chamber two containing it just to cover the entire surface of the ulcer.
5) Acetic acid is sprayed again from the chamber one on the surface of the ulcer to wash the unabsorbed TBO.
6) Only the malignant/cancerous tissue takes up the TBO formulation and looks dark blue. Further, clinical diagnosis of oral cancer can be made. For confirmation, the colored tissue can be biopsied and microscopic evaluation may be performed. Benign tissue (non-malignant/non-cancerous tissue) will not take up TBO formulation and hence will not stain dark blue.

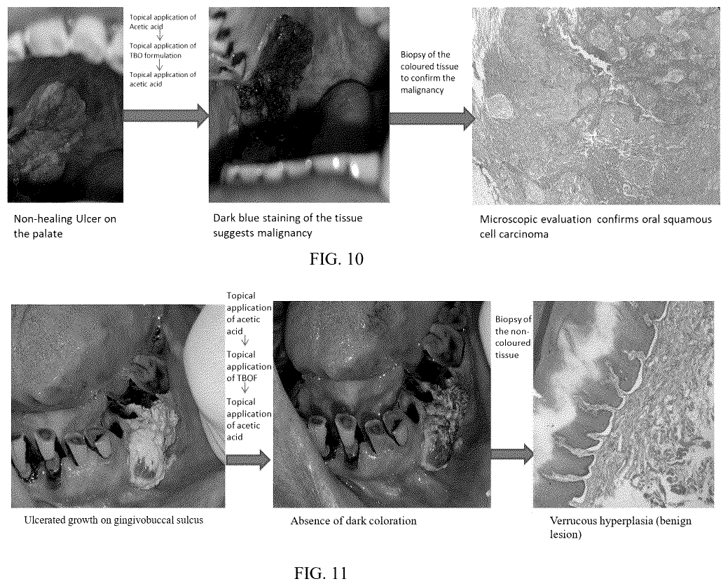

FIG. 10 demonstrates positive results after screening, appreciated as dark blue color stained tissue after the application of the kit protocol. FIG. 11 demonstrates negative results after screening, appreciated as absence of dark blue color stained tissue after the application of the kit protocol.

To evaluate the screening potential of the two formulations, vital stain formulation formulation and food debris cleaning solution was topically applied with the help of dual chamber sprayer in 365 patients who were diagnosed clinically for oral cancer.

The staining was evident in 295 patients. The stained and unstained tissue were identified by the naked eye, were biopsied and sent for histopathological diagnosis. All 295 tissues were diagnosed as positive for oral cancer. The staining was not evident in 70 patients. Lesions from these 70 cases were biopsied through the conventional clinician's visual site determination. Upon histopathological examination, 50 cases were proven as non-malignant lesions (benign) and 20 cases were found to be malignant.

Thus, following results were obtained:
True positive-295
True negative-50
False positive-0
False negative-20
True positive rate/sensitivity=93.65%
True negative rate (specificity)=100%

Thus, advantageously the Toluidine blue O synthesized by the method described in the present invention produces more intense coloration while staining the malignant tissues. The purity of Toluidine blue O synthesized by the method described in the present invention is acceptable by the regulatory bodies and is simple to reproduce by pharmaceutical manufacturers. The vital stain formulation in accordance with the present invention is useful for identification of biopsy sites in patients diagnosed clinically for oral cancer. The kit in accordance with the present invention, is ready to use and has vital stain formulation with increased shelf life and stability. The formulations have improved palatability and acceptability among patients and dental surgeons. The formulations reduces false positive results as seen in Laboratory grade Toluidine blue O.

Advantageously, the protocol of sequential delivery of the formulations via the kit is easy and convenient for chair side procedure. The present invention also helps to detect innocuous secondary oral cancer while examining the patients treated for oral cancer during follow up visits. The kit offers a cost effective ready to use tool for oral cancer screening. Because of the purest form, the vital stain formulation of Toluidine blue O synthesized by the process of the present invention can be used as an effective photosensitizer during photodynamic therapy.

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the scope of the present invention.

1. A process 100 for the synthesis of Toluidine blue O, comprising:
(a) a first stage 105 of synthesizing of 2-amino-5-dimethylaminophenyl thiosulfonic acid;
(b) a second stage 110 of synthesizing of indamine thiosulfonic acid;
(c) a third stage 115 of synthesizing Toluidine blue O and Toluidine blue O Zinc Chloride Double Salt; and
(d) a fourth stage 120 of purifying Toluidine blue O.

2. The process as claimed in claim 1, wherein the first stage 105 of synthesizing 2-amino-5-dimethylaminophenyl thiosulfonic acid includes:
(a) a first step of adding hydrochloric acid to a stirred solution of N, N-dimethyl-1,4-phenylenediamine in water and stirring for 15 mins to obtain a reaction mass of pH 2-3;
(b) a second step of adding a solution of aluminum sulfate hexadecahydrate in water to the above mixture of first step and stirring at a room temperature for 10 mins;
(c) a third step of adding a solution of Zinc chloride in water to the above mixture of second step and stirring at a room temperature for 5 mins, and cooling the reaction mass further to 4° C.;
(d) a fourth step of adding a solution of potassium dichromate in water dropwise and stirring for 20 mins, followed by bringing the reaction mass to room temperature;
(e) a fifth step of adding a solution of sodium thiosulfate pentahydrate in water at room temperature and stirring for 30 mins; and
(f) a sixth step of heating the reaction mass to 60° C. and stirring for 5 mins, and bringing the reaction again to room temperature.

3. The process as claimed in claim 1, wherein the second stage 110 of synthesizing of indamine thiosulfonic acid includes:
(a) a first step of adding a solution of o-toluidine in water to the reaction mass obtained in the sixth step of first stage 105 at 10° C. and stirring for 5 mins; and
(b) a second step of adding a solution of potassium dichromate in water dropwise, and stirring for 10 mins, followed by stirring the reaction mass at 60° C. for 25 mins.

4. The process as claimed in claim 1, wherein the third stage 115 of synthesizing Toluidine blue O and Toluidine blue O Zinc Chloride Double Salt includes:
(a) a first step of cooling the reaction mixture obtained in second step of the second stage 110 to 0° C., followed by adding solution of potassium dichromate in water dropwise to it and stirring for 20 mins;
(b) a second step of again adding a solution of potassium dichromate in water dropwise to it and stirring for 20 mins;
(c) a third step of adding a solution of zinc chloride in water to the above mixture obtained in second stage at 10° C. and stirring at room temperature for 20 mins;
(d) a fourth step of adding a solution of copper sulfate pentahydrate in water to the rection mass obtained in above third step, and stirring for 15 mins; followed by heating the reaction mass at 60° C.; and at this temperature, adjusting the pH of the reaction mass to 2-3 using sulfuric acid solution; and
(e) a fifth step of raising the temperature of the reaction mass to 100° C. and stirring for 35 mins, and after 1 hr, cooling the reaction to 0° C. and keeping for 16 hrs.

5. The process as claimed in claim 1, wherein the fourth stage 120 of purifying Toluidine blue O includes:
(a) a first step of filtering the reaction mass obtained in the fifth step of third stage 115, over a Buchner funnel and discarding the filtrate;
(b) a second step of washing the solid cake with aqueous sodium chloride solution and discarding the filtrate;
(c) a third step of redissolving the solid residue in 100 ml water and stirring for 4 hrs at room temperature;
(d) a fourth step of heating the reaction at 75° C., and once that temperature is reached, bringing the reaction to 40° C. and stirring for 16 hrs;
(e) a fifth step of cooling the reaction, and filtering and discarding the undissolved particles;
(f) a sixth step of adding zinc chloride to the filtrate for precipitation and again cooling to 0° C.;
(g) a seventh step of redissolving the obtained Toluidine blue O (TBO) in MeOH and precipitating it out with Methyl tertbutylether as a co-solvent to get TBO;
(h) an eighth step of crystallizing, followed by drying and obtaining a yield of Toluidine blue O as an amorphous powder weighing 0.7 g; and
(i) a ninth step of characterizing the purity, elution time, area covered and peak intensities of Toluidine blue O and its derivatives by LCMS and NMR.

6. A kit for screening and biopsy site identification in oral cancer lesions with convenience, based on Toluidine blue O synthesized by the process as claimed in claim 1, wherein the kit including
(a) a first formulation of a food debris cleaning solution containing 1% flavored acetic acid,
(b) a second formulation of vital stain formulation including
i. Toluidine blue O: 0.5-1 g,
ii. Potassium Iodide: 90-100 mg,
iii. Ascorbic Acid: 60-65 mg,
iv. Acetic Acid: 3-5 g,
v. Ethanol: 7-10 ml,
vi. Menthol: 0.2-0.4 g, and
vii. Water added to make 100 ml.
the formulations being dispensed through a dual chamber sprayer.

7. The kit for screening and biopsy site identification in oral cancer lesions as claimed in claim 6 wherein,
a first chamber containing the first formulation and a second chamber containing the second formulation, and
the protocol including spraying the first formulation of cleaning solution from the first chamber of the dual chamber sprayer on the surface of the oral lesion for removing any debris, followed by spraying the second formulation of vital stain from the second chamber on the surface of the oral lesion, and spraying the first formulation of cleaning solution from the first chamber on the surface of the oral lesion for de-staining the normal cells.

8. The second vital stain formulation as claimed in claim 6, wherein the formulation is stable even after three months of storage; and having C15H16N3S([MS]+) of 270.06 that is tested after three months of storage with the help of ESI Mass analysis.
